(19) United States
(12) Patent Application Publication
(10) Pub. No.: US 2019/0314449 A1
(43) Pub. Date: Oct. 17, 2019

(54) CYCLIC DIPEPTIDES AND WOUND HEALING

(71) Applicant: Anand PRASAD, San Antonio, TX (US)

(72) Inventor: Anand PRASAD, San Antonio, TX (US)

(73) Assignee: THE BOARD OF REGENTS OF THE UNIVERSITY OF TEXAS SYSTEM, Austin, TX (US)

(21) Appl. No.: 16/461,084

(22) PCT Filed: Nov. 16, 2017
(86) PCT No.: PCT/US2017/061977

§ 371 Date: May 15, 2019

Related U.S. Application Data
(60) Provisional application No. 62/422,928, filed on Nov. 16, 2016.

(51) Int. Cl. A61K 38/12; A61K 9/00; A61K 38/05; A61P 17/02

(52) U.S. Cl. CPC A61K 38/12; A61P 17/02; A61K 38/05; A61K 9/0014

(57) ABSTRACT

A pharmaceutical composition comprising a cyclo-dipeptide and use thereof for treatment of a wound. The cyclo-dipep-tide can be Cyclo-His-Pro and the pharmaceutical composition can suitable for topical application to a wound. The compositions can be used to treat a wound, reduce tissue damage in a wound, prevent and/or reduce oxidative damage in a wound, and/or reduce the amount of heme in a wound.

This Application claims priority to U.S. Provisional Patent Application Ser. No. 62/422,928 filed Nov. 16, 2016, which is incorporated herein by reference in its entirety.

### BACKGROUND

Chronic wounds impact 1% of the population in industrialized nations and consume 2-4% of healthcare expenditures. Chronic wounds are wounds that do not heal in an expected amount of time and/or in an expected order or stages. Typically, a wound that does not heal in three months is considered a chronic wound, but a wound may be considered chronic well before the three month threshold. Some examples of chronic wounds include arterial ulcers, neuropathic ulcers, pressure sores, and venous ulcers. Various factors can contribute to the development of a wound into a chronic wound. Predisposing factors include venous insufficiency, arterial insufficiency, diabetes, neuropathy, renal impairment, systemic morbidity that includes fibrosis, atherosclerosis, edema, sickle cell disease, malignancy, lymphoedema, trauma, ischemia reperfusion injury, rheumatological morbidity, malnutrition, neuropathy, vasomotor paralysis, pressure over prominent bone, use of corticosteroids, vasculitis, plaque instability, thrombosis, immune suppression, pyoderma gangrenosum, and infection.

The pathophysiology of chronic wounds is complex and can be complicated by dyslipidemia, increased inflammation, hyperglycemia, increased advanced glycation end products, impairment of angiogenesis, and increased reactive oxygen species. In chronic wounds, as well as non-chronic wounds, tissue damage can be increased and/or perpetuated by release of heme into the wound. When blood vessels are disrupted, such as through tissue injury or leaking vessels, free heme from the blood can leak into the surrounding tissue. This free heme is toxic to the surrounding tissues and causes tissue damage because of its ability to cause oxidative damage, cross-linking, aggregation, and degradation of biomolecules including proteins, lipids, and DNA. Free heme can also activate inflammation pathways in the tissue which contributes to tissue damage.

The first line standardized clinical treatments for chronic wound care is preparation and direct treatment of the wound bed to assist in the healing of the wound. Additional treatment options include surgical interventions, such as skin grafts, and addressing the underlying pathology, such as management of diabetes. Wound bed preparation and treatment generally focuses on debridement, bacterial load management, and keeping the wound bed moist. However, there are limited topical agents for wound healing and those that are available are expensive, have variable efficacy, and/or may have not undergone sufficient randomized trials to demonstrate statistically significant efficacy. Thus, it is difficult to know which topical treatment has the greatest likelihood of assisting the healing of a patient's chronic wound. Low cost and effective treatments for wounds are needed.

### SUMMARY

There is a need for development of a low-cost and effective treatment for wounds. A solution to the aforementioned problems associated with treatments for wounds has been discovered. The solution is premised on the use of one or more cyclic dipeptide(s) (CDP). Not to be bound by theory, it is believed that CDPs may be potent anti-oxidants that help wound healing by limiting tissue damage. In some embodiments CDP(s) include: Cyclo-Ala-Pro; Cyclo-Arg-Pro; Cyclo-Asn-Pro; Cyclo-Asp-Pro; Cyclo-Cys-Pro; Cyclo-Glu-Pro; Cyclo-Gly-Pro; Cyclo-His-Pro; Cyclo-Ile-Pro; Cyclo-Leu-Pro; Cyclo-Lys-Pro; Cyclo-Met-Pro; Cyclo-Phe-Pro; Cyclo-Pro-Pro; Cyclo-Ser-Pro; Cyclo-Thr-Pro; Cyclo-Trp-Pro; Cyclo-Tyr-Pro; Cyclo-Val-Pro; and/or Cyclo-Gln-Pro. In an preferred embodiment, the CDP(s) include Cyclo-His-Pro (CHP).

Disclosed herein is a wound care composition containing CDP having the general structure of Formula I:

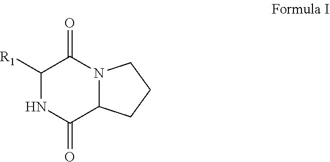

where R₁ is any amino acid side chain. In certain aspects the amino acid side chain is an alanine, arginine, asparagine, aspartic acid, cysteine, glutamine, glutamic acid, glycine, histidine, isoleucine, leucine, lysine, methionine, phenylalanine, serine, threonine, tryptophan, tyrosine, or valine side chain.

Disclosed herein is a wound care composition containing Cyclo-His-Pro having the structure of Formula II:

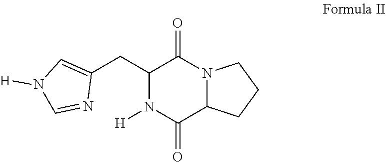 [SMILES: O=C1NC(Cc2c[nH]cn2)C(=O)N2CCCC12]

In some aspects, the composition contains an effective amount of Cyclo-His-Pro to treat a wound. In certain aspects treating a wound includes one or more of providing an antioxidant to a wound, reducing the amount of heme in a wound, inducing heme oxygenase-1 expression in a wound, decreasing the amount of tissue damage in a wound, and/or activating nuclear factor, erythroid 2 like 2 (Nrf2) in a wound or cells contained within or surrounding a wound. A cell surrounding a wound can be a cell that is within 0.1 to 2 mm of a break in the skin.

In some aspects the composition further comprises a pharmaceutically acceptable carrier. The pharmaceutically acceptable carrier can be a naturally occurring or a non-naturally occurring carrier.

In some aspects, the composition is formulated for topical application onto a wound. In some instances, the composition is formulated as an emulsion. In some instances, the composition is formulated as an ointment, a balm, a serum, a cream, a foam, a film, an aqueous liquid, or an oil based liquid.

A CDP, e.g., Cyclo-His-Pro, can be the only active ingredient in the composition or the composition can contain additional active ingredients. In some instances, the composition contains one or more additional active ingredient(s) capable of moisturizing, capable of increasing cellular uptake of compounds, capable of increasing neovascularization, capable of providing anti-microbial activity, capable of elevating nitrogen monoxide levels, capable of increasing vascular endothelial growth factor (VEGF) activity and/or concentration, capable of enhancing re-epithelialization, and/or capable of decreasing inflammation. In certain aspects the active ingredients are provided in a hydrogel. In some instances an additional active ingredient includes Cyclo-Leu-Pro, Cyclo-Phe-Pro, chitosan, zinc oxide, and/or arginine. In some instances, the composition contains an effective amount of a hydrogel to increase cellular uptake and/or increase neovascularization. In some instances, the composition contains an effective amount of Cyclo-Leu-Pro to provide anti-microbial activity. In some instances, the composition contains an effective amount of Cyclo-Phe-Pro to provide anti-microbial activity. In some instances, the composition contains an effective amount of chitosan to provide anti-microbial activity. In some instances, the composition contains an effective amount of zinc oxide to provide anti-microbial activity, to enhance re-epithelialization, and/or to decrease inflammation. In some instances, the composition contains an effective amount of arginine to elevate nitrogen monoxide levels and/or increase Vascular Endothelial Growth Factor (VEGF) activity and/or concentration.

In some aspects, the composition can further comprise one or more additional cyclic dipeptide(s). The cyclic dipeptide(s) can include, but are not limited to, cyclic dipeptide(s) containing proline as one of the two amino acids. In some instances, the one or more additional cyclic dipeptide(s) include Cyclo-Ala-Pro; Cyclo-Arg-Pro; Cyclo-Asn-Pro; Cyclo-Asp-Pro; Cyclo-Cys-Pro; Cyclo-Glu-Pro; Cyclo-Gly-Pro; Cyclo-Ile-Pro; Cyclo-Leu-Pro; Cyclo-Lys-Pro; Cyclo-Met-Pro; Cyclo-Phe-Pro; Cyclo-Pro-Pro; Cyclo-Ser-Pro; Cyclo-Thr-Pro; Cyclo-Trp-Pro; Cyclo-Tyr-Pro; Cyclo-Val-Pro; and/or Cyclo-Gln-Pro.

Also disclosed herein is a wound care composition containing one or a combination of cyclic dipeptide(s). In some instances, the composition further contains a pharmaceutically acceptable carrier. The pharmaceutically acceptable carrier can be a naturally occurring or a non-naturally occurring carrier. One or more of the cyclic dipeptides contained therein can, but are not limited to, proline containing cyclic dipeptides. In some instances, the cyclic dipeptide(s) is/are one or a combination of Cyclo-Ala-Pro; Cyclo-Arg-Pro; Cyclo-Asn-Pro; Cyclo-Asp-Pro; Cyclo-Cys-Pro; Cyclo-Glu-Pro; Cyclo-Gly-Pro; Cyclo-His-Pro; Cyclo-Ile-Pro; Cyclo-Leu-Pro; Cyclo-Lys-Pro; Cyclo-Met-Pro; Cyclo-Phe-Pro; Cyclo-Pro-Pro; Cyclo-Ser-Pro; Cyclo-Thr-Pro; Cyclo-Trp-Pro; Cyclo-Tyr-Pro; Cyclo-Val-Pro; and/or Cyclo-Gln-Pro.

In some aspects, the composition is formulated for topical application onto a wound. In some instances, the composition is formulated as an emulsion. In some instances, the composition is formulated as an ointment, a balm, a serum, a cream, a foam, a film, an aqueous liquid, or an oil based liquid.

The cyclic dipeptide(s) can be the only active ingredient or ingredients in the composition or the composition can contain additional active ingredients. In some instances, the composition contains one or more additional active ingredient(s) capable of moisturizing, capable of increasing heme-scavenging, capable of increasing cellular uptake of compounds, capable of increasing neovascularization, capable of providing anti-microbial activity, capable of elevating nitrogen monoxide levels, capable of increasing Vascular Endothelial Growth Factor (VEGF) activity and/or concentration, capable of enhancing re-epithelialization, and/or capable of decreasing inflammation. In some instances the additional active ingredient is a hydrogel, Cyclo-His-Pro, Cyclo-Leu-Pro, Cyclo-Phe-Pro, chitosan, zinc oxide, and/or arginine. In some instances, the composition contains an effective amount of hydrogel to increase cellular uptake and/or increase neovascularization. In some instances, the composition contains an effective amount of Cyclo-His-Pro to increase heme-scavenging. In some instances, the composition contains an effective amount of Cyclo-Leu-Pro to provide anti-microbial activity. In some instances, the composition contains an effective amount of Cyclo-Phe-Pro to provide anti-microbial activity. In some instances, the composition contains an effective amount of chitosan to provide anti-microbial activity. In some instances, the composition contains an effective amount of zinc oxide to provide anti-microbial activity, to enhance re-epithelialization and/or decrease inflammation. In some instances, the composition contains an effective amount of arginine to elevate nitrogen monoxide levels and/or increase Vascular Endothelial Growth Factor activity and/or concentration.

Methods of using any one or a combination of the compositions disclosed herein are disclosed. Methods of treating a wound, inducing heme oxygenase-1 expression in a wound, activating Nuclear factor, erythroid 2 like 2 (Nrf2) in a wound, reducing tissue damage in a wound, preventing and/or reducing oxidative damage in a wound, and/or reducing the amount of heme in a wound are disclosed. In some instances, any of the methods disclosed herein include administering any one of the compounds and/or compositions disclosed herein to the wound. In some instances, any of the methods disclosed herein include administering the composition topically to the wound. In some instances, the wound is a chronic wound or a non-chronic wound. In some instances, the wound is a venous, arterial, diabetic, burn, and/or pressure wound.

In certain aspects the compositions and compounds described herein can be administered to attenuate scar formation or scarring. The scarring can be associated with a wound or fibrotic disorder. A therapeutically effective amount of a compound or composition described herein is an amount of compound or composition that is able to inhibit scarring of a wound (or site at which a wound is to be formed) or a fibrotic disorder (or site at which a fibrotic disorder will occur) to which the compound or composition is administered. A therapeutically effective amount of a medicament of the invention is any amount of a medicament of the invention that is able to inhibit scarring. This inhibition of scarring may preferably be achieved at a site to which the medicament of the invention is administered. A therapeutically effective amount can be an amount that is effective to inhibit scarring by at least 10, 20, 30, 40, 50, 60, 70, 80, 90% compared to a relevant control.

A scar may be defined as “fibrous connective tissue that forms at the site of injury or disease in any tissue of the body” (the scarring response is common throughout all adult mammals). Scarring may result from healing of a wound, or through the deposition of scar tissue associated with fibrotic disorders. The scarring response is conserved between the majority of tissue types and in each case leads to the same result, formation of fibrotic tissue termed “a scar”. Many different processes are at work during the scarring response, and much research has been conducted into discovering what mediates these processes, and how they interact with each other to produce the final outcome.

The scarring response has arisen as the evolutionary solution to the biological imperative to prevent the death of a wounded animal. Thus, to overcome the risk of mortality due to infection or blood loss, the body reacts rapidly to repair the damaged area, rather than attempt to regenerate the damaged tissue.

In the case of a scar that results from healing of a wound, the scar may be defined as the structure produced as a result of the reparative response. Since the injured tissue is not regenerated to attain the same tissue architecture present before wounding, a scar may be identified by virtue of its abnormal morphology as compared to unwounded tissue. Such scars are composed of connective tissue deposited during the healing process. A scar may comprise connective tissue that has an abnormal organization (as seen in scars of the skin) and/or connective tissue that is present in an abnormally increased amount. Most scars consist of both abnormally organized and excess connective tissue.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, devices, compositions, and kits of the invention can be used to achieve methods of the invention.

The term “active ingredient” or “active ingredients”, as used herein, includes any compound or therapeutic agent known to or that demonstrates advantageous properties when administered to a subject in need of treatment for a wound, wound healing.

The term “pharmaceutically acceptable” when used in this application means acceptable for use in a pharmaceutical product. Pharmaceutically acceptable typically means non-toxic when administered to or contacting a subject and non-reactive with the active ingredients in the pharmaceutical product.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. In some instances, the carrier is a naturally derived carrier. In some instances, the carrier is non-naturally derived.

The term “pharmaceutically acceptable carrier” when used in this application means a carrier acceptable for use in a pharmaceutical composition for administration to a subject. Pharmaceutically acceptable carriers are typically non-toxic. In some instances, the pharmaceutically acceptable carrier is a naturally derived carrier. In some instances, the pharmaceutically acceptable carrier is non-naturally derived.

The use of the word “a” or “an” when used in conjunction with the term “comprising” in the claims and/or the specification may mean “one,” but it is also consistent with the meaning of ‘one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Compositions and methods containing one or more cyclic dipeptide(s) useful in treating and/or preventing wounds are disclosed herein. Some embodiments of these compositions and methods have the advantages of being low-cost and/or effective for topical administration.

Not to be bound by theory, it is believed that CDPs may be potent anti-oxidants that help wound healing by limiting tissue damage. Specifically, CPDs may protect against oxidative damage by induction of antioxidative genes through selectively activating the transcription factor, Nuclear factor, erythroid 2 like 2 (Nrf2). Nrf2 binds to the antioxidant response element (ARE) in the promoter region of antioxidative genes and increases expression of these genes. Heme oxygenase-1 (HO-1) is a protein induced by Nrf2 and is the rate-limiting enzyme in the degradation of heme into carbon monoxide, iron, and biliverdin, which is subsequently reduced to bilirubin. HO-1 has been shown to be upregulated following cutaneous injury and has been localized to the granulation tissue of wounds (Hanselmann et al. Biochem. J. (2001) 353:459-466). Thus, not to be bound by theory, it is believed that CDPs can increase the rate of wound healing and decrease the rate of tissue damage through the induction of HO-1.

### I. Cyclic Dipeptides (CDP(s))

Cyclic dipeptide are compounds containing two amino acids covalently bound in a ring structure through two or more peptide bonds. Many cyclic dipeptides are found in foods that undergo processing that involves enzymatic hydrolysis and/or thermal processing.

CDP(s) can include combinations of any two amino acids, including, but not limited to combinations of the twenty standard amino acids. In some embodiments, at least one of the two amino acids in the CDP is proline. In some embodiments, the CDP is Cyclo-Ala-Pro, Cyclo-Arg-Pro, Cyclo-Asn-Pro, Cyclo-Asp-Pro, Cyclo-Cys-Pro, Cyclo-Glu-Pro, Cyclo-Gly-Pro, Cyclo-His-Pro, Cyclo-Ile-Pro, Cyclo-Leu-Pro, Cyclo-Lys-Pro, Cyclo-Met-Pro, Cyclo-Phe-Pro, Cyclo-Pro-Pro, Cyclo-Ser-Pro, Cyclo-Thr-Pro, Cyclo-Trp-Pro, Cyclo-Tyr-Pro, Cyclo-Val-Pro; and/or Cyclo-Gln-Pro. In an preferred embodiment, the CDP(s) include Cyclo-His-Pro.

Herein, it is disclosed that CDP(s) can be used to treat wounds and can be effective in topical applications. CDP(s) can be used to treat many different types of wounds, including venous, arterial, diabetic, burn, and pressure.

A. Cyclo-His-Pro (CHP)

Cyclo-His-Pro is a CDP that contains histidine and proline and has the structure shown in Formula II.

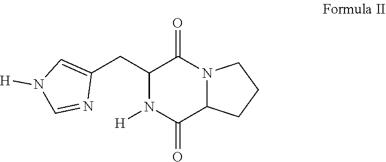 [SMILES: O=C1NC(Cc2c[nH]cn2)C(=O)N2CCCC12]

CHP has been shown to be safe for human use. CHP is endogenous to a variety of human tissues, body fluids, and foods that humans routinely consume. CHP is common to many foods that undergo processing that involves enzymatic hydrolysis and/or thermal processing. Further, CHP has been used in food supplements and is suspected of being contained in some food sources such as tuna, yogurt, and ham. (Hilton et al. Lancet. 1990; 336(8728):1455; Hilton et al. J Clin Endocrinol Metab. 1992; 75(2):375-378). In addition, a human study on the pharmacokinetics and possible toxicity of acute oral consumption of synthetic CHP (24 mg/dose) showed that the peptide is absorbed from the gastrointestinal tract, with plasma CHP levels peaking at 4 h and returning to normal by 24 h. Uyemura et al. J. Drug Metab. Toxicol. 2010; 1(2):105. No side effects were experienced in any of the subjects based on physical and blood chemistry examinations. Id.

CHP has been suggested to up-regulate heme oxygenase 1 via activation of Nrf-2 ARE signaling in neuronal cultures. (Minelli et al. J. Neurochem. 2009; 111: 956-966).

Herein, it is disclosed that CHP can be used to treat wounds and can be effective in topical applications. CHP can be used to treat many different types of wounds, including venous, arterial, diabetic, burn, and pressure.

### II. Additional Active Ingredients

The CDP(s) disclosed herein can be used as the only active ingredient in a pharmaceutical administered to a subject or can be combined with additional active ingredients. In some embodiments the additional active ingredients include agents capable of moisturizing, decreasing heme concentration, increasing cellular uptake of compounds, increasing neovascularization, providing anti-microbial activity, elevating nitrogen monoxide levels, increasing Vascular Endothelial Growth Factor activity and/or concentration, enhancing re-epithelialization, and/or decreasing inflammation. In some instances, the agent capable of decreasing heme concentration is Cyclo-His-Pro, increasing cellular uptake of compounds is a hydrogel, increasing neovascularization is a hydrogel, providing anti-microbial activity is Cyclo-Leu-Pro, Cyclo-Phe-Pro, chitosan, and/or zinc oxide, elevating nitrogen monoxide levels is arginine, increasing Vascular Endothelial Growth Factor activity and/or concentration is arginine, enhancing re-epithelialization is zinc oxide, and/or decreasing inflammation is zinc oxide.

### III. Compositions

It is contemplated that the compositions of the present invention can include any amount of the ingredients discussed in this specification. The compositions can also include any number of combinations of additional ingredients described throughout this specification (e.g., stabilizers, fillers, pharmaceutically and/or nutraceutical acceptable salts, and/or additional pharmaceutical and/or nutraceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition or relative abundance. A person of ordinary skill in the art would understand that the concentrations can vary depending on the addition, substitution, and/or subtraction of ingredients in a given composition.

### IV. Additional Components

The compositions and compounds of the present invention can be formulated into any suitable composition form for administration to a human or a non-human animal subject. The composition may consist of the claimed compound or compounds alone or may include the compound or compounds and any suitable additional component, such as one or more pharmaceutically and/or nutraceutical acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient.

1. Excipients

Excipients employed in the compositions of the present invention can be solids, semi-solids, liquids or combinations thereof. Compositions of the invention containing excipients can be prepared by any known technique that comprises, for example, admixing an excipient with the claimed compounds and/or compositions. A pharmaceutical composition of the invention contains a desired amount of the claimed compounds per dose unit.

2. Carriers/Diluents

Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, can constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

3. Disintegrant

Compositions of the invention optionally can include one or more pharmaceutically and/or nutraceutical acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, can constitute in total about 0.2% to about 30%, preferably about 0.2% to about 10%, and more preferably about 0.2% to about 5%, of the total weight of the composition.

4. Binders

The compositions of the present invention can include binding agents or adhesives particularly for binding to a surface or into a tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, HPMC, hydroxypropylcellulose, and ethylcellulose. Such binding agents and/or adhesives, if present, can constitute in total about 0.5% to about 25%, preferably about 0.75% to about 15%, and more preferably about 1% to about 10%, of the total weight of the pharmaceutical composition. Many of the binding agents are polymers comprising amide, ester, ether, alcohol or ketone groups and, as such, can be included in pharmaceutical compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units’ ratios in a given polymer affects properties and performance.

5. Wetting Agents

Wetting agents can be used in the compositions of the present invention. Wetting agent can be selected to maintain the a crystal in close association with water, a condition that may improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, can constitute in total about 0.25% to about 15%, preferably about 0.4% to about 10%, and more preferably about 0.5% to about 5%, of the total weight of the pharmaceutical composition.

6. Lubricants

Lubricants can be included in the compositions of the present invention. Suitable lubricants include, but are not limited to, either individually or in combination, glyceryl behapate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, can constitute in total about 0.1% to about 10%, preferably about 0.2% to about 8%, and more preferably about 0.25% to about 5%, of the total weight of the composition.

7. Other Agents

Surfactant, emulsifier, or effervescent agents can be used in the compositions. Emulsifying agents can be used to help solubilize the ingredients within a composition. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

8. Vehicles

Various delivery systems are known in the art and can be used to administer a therapeutic agent or composition of the invention, e.g., encapsulation in liposomes, microparticles, microcapsules, receptor-mediated endocytosis and the like. Methods of administration include, but are not limited to, topical, parenteral, intra-arterial, intramuscular, intravenous, intranasal, and oral routes. The compositions can be provided in the form of suspensions, solutions, and emulsions of the active ingredient in aqueous or non-aqueous diluents, syrups, granulates, or powders. The compositions can also be provided in the form of tablets, lozenges, granules, capsules, pills, ampoule, suppositories or aerosol form.

V. Formulations and Administration

One non-limiting benefit of embodiments described herein is that therapeutic compositions can be delivered and have effect quickly and easily. The disclosed compositions can be formulated as pharmaceutical compositions for the administration to a subject. Pharmaceutical compositions may be administered, but are not limited to, via topical application. In certain aspects the compositions are deposited on a wound by methods and devices known in the art. Compositions may be prepared in water suitably mixed with a surfactant, such as hydroxypropylcellulose. Compositions may also be prepared in glycerol, liquid polyethylene glycols and mixtures thereof, and in oils. Under ordinary conditions of storage and use, these preparations contain a preservative to prevent the growth of microorganisms. Formulations suitable for topical administration according to the subject invention can be formulated as, but is not limited to, an ointment, cream, suspension, lotion, powder, solution, paste, gel, spray, aerosol, or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients into the skin. The compositions can be formulated to be stable under the conditions of manufacture and storage and can be preserved against the contaminating action of microorganisms, such as bacteria and fungi. The carrier can be a solvent or medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like.

Some variation in dosage will necessarily occur depending on the condition of the subject being treated and the particular circumstances involving exposure or potential exposure. The person responsible for administration will, in any event, determine the appropriate dose for the individual subject. Moreover, for human administration, preparations can meet sterility, pyrogenicity, general safety, and purity standards as required by FDA standards or other similar organizations.

Sterile compositions can be prepared by incorporating the active components in the required amount in the appropriate solvent with various other ingredients enumerated above, as required, followed by, for example, filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some methods of preparation are vacuum-drying and freeze-drying techniques which yield a powder of the active ingredient plus any additional desired ingredient from a previously sterile-filtered solution.

A spray comprising a pharmaceutical composition can be produced by forcing a suspension or solution of a composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

Methods may involve administering to the patient or subject at least or at most 1, 2, 3, 4, 5, 6, 7, 8, 9, 10 or more doses of a therapeutic composition. A dose may be a composition comprising about, at least about, or at most about 0.01, 0.05, 0.1, 0.5, 1.0, 5.0, 10.0, 15.0, 20.0, 50, 100, 200, 300, 400, 500, 600, 700, 800, 900, 1000 milligrams (mg) or micrograms (mcg) or μg/ml or micrograms/ml or mM or μM (or any range derivable therein) of CDP(s) or the total amount of a combination of CDP(s).

A dose may be administered on an as needed basis or every 1, 2, 3, 4, 5, 6, 7, 8, 9, 10, 11, 12, 18, or 24 hours or days (or any range derivable therein) or 1, 2, 3, 4, 5, 6, 7, 8, 9, or times per day or week (or any range derivable therein). A dose may be first administered before or after signs of a wound are exhibited or felt by a patient or after a clinician evaluates the patient for a wound. In some embodiments, the patient is administered a first dose of a regimen 1, 2, 3, 4, 5, 6, 7, 8, 9, 10, 11, 12 hours (or any range derivable therein) or 1, 2, 3, 4, or 5 days, weeks, or months after the patient experiences or exhibits signs or symptoms of a wound (or any range derivable therein). The patient may be treated for 1, 2, 3, 4, 5, 6, 7, 8, 9, 10 or more days, weeks, or months (or any range derivable therein) or until the wound has disappeared or been reduced or after 6, 12, 18, or 24 hours or 1, 2, 3, 4, or 5 days, weeks, or months after the wound has disappeared or been reduced.

In compositions comprising two CDA(s), the ratio of the two CDA(s) may be about 1:1, 2:1, 3:1, 4:1, 5:1, 6:1, 7:1, 8:1, 9:1, 10:1, 11:1, 12:1, 13:1, 14:1, 15:1, 20:1, 30;1, 40:1, 50:1, 60:1, 70:1, 80:1, 90:1, 100:1 or more, or any range derivable therein.

Treatment with a composition disclosed herein can be at least or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, or 120 minutes or hours in length (or any value or range there between). The composition may contain about, at least about or at most about 0.1, 1, 10, 50, 100, 150, 200, 250, 300, 400, 500, 600, 700, 800, 900, 1000 μg/ml or mg/ml (or any value or range there between) of each CDP(s) or the total amount of a combination of CDPs.

The volume that is administered in each dose may be about, at least about, or at most about 0.01, 0.05, 0.1, 1.0, 2.0, 3.0, 4.0, 5.0, 6.0, 7.0, 8.0, 9.0, 10.0, 15.0, 20.0. 25.0, 30.0, 35.0, 40.0, 45.0, 50.0, 55.0, 60.0, 65.0, 70.0, 75.0, 80.0, 85.0, 90.0, 95.0, 100.0 μl or ml (or any value or range there between).

### VI. Secondary or Combination Treatments

Certain embodiments provide for the administration or application of one or more secondary or additional forms of therapies or preventative interventions. The type of therapy is dependent upon the type of wound and/or disease that is being treated or prevented. The secondary form of therapy may be administration of one or more secondary pharmacological agents that can be applied in the treatment or prevention of a disease and/or condition.

If the secondary or additional therapy is a pharmacological agent, it may be administered prior to, concurrently, or following administration of the compound and/or compositions disclosed herein. The interval between administration of the compound and/or compositions disclosed herein and the secondary or additional therapy may be any interval as determined by those of ordinary skill in the art. For example, the compound and/or compositions disclosed herein and the secondary or additional therapy may be administered simultaneously, or the interval between treatments may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, it may be desirable to extend the time period for treatment significantly, such as where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the compound and/or compositions disclosed herein.

### VII. Kits

In another aspect of the present invention, kits for treating or preventing a disease, condition, or disorder as described herein. For instance, compositions of the present invention can be included in a kit. A kit can include a container. Containers can include a bottle, a metal tube, a laminate tube, a plastic tube, a dispenser, a straw, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a predetermined amount of the composition. In other embodiments, the container can be squeezed (e.g., metal, laminate, or plastic tube) to dispense a desired amount of the composition. The composition can be dispensed as a spray, an aerosol, a liquid, a fluid, a semi-solid, or a solid. In a preferred embodiment, the composition is dispensed as a composition capable of being topically applied. The containers can have spray, pump, or squeeze mechanisms. A kit can also include instructions for employing the kit components as well the use of any other compositions included in the container. Instructions can include an explanation of how to apply, use, and maintain the compositions. The compositions can, if desired, be presented in a pack or dispenser device, which can contain one or more unit dosage forms containing the compound compositions. The pack can, for example, comprise metal or plastic foil, such as a blister pack. The pack or dispenser device can be accompanied by instructions for administration.

### EXAMPLES

The following examples are included to demonstrate preferred embodiments of the invention. It should be appreciated by those of skill in the art that the techniques disclosed in the examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Herein is disclosed that treatment of animal wound models with topically applied Cyclo-His-Pro significantly reduce in vivo wound healing time. Further, dosages of Cyclo-His-Pro has also been determined that are useful in those models. The results indicate that Cyclo-His-Pro have a broad wound healing activity spanning different types of wounds and are applicable to other species of subjects, such as human subjects.

Compositions suitable for topical application to a subject can be formulated and used in the experiments disclosed herein.

A topical composition that provides multiple benefits when applied topically to a subject can be formulated to include one or more active ingredient selected form Cyclo-His-Pro, Cyclo-Leu-Pro, Cyclo-Phe-Pro, or other CDP; hydrogel; chitosan; zinc oxide; and arginine. Examples of formulations with variable dosages of Cyclo (His-Pro) are shown in Table 1. The dosages will consist of low dose and high dose.

TABLE 1

| CDP | + Inert agents | +Zn | +Arginine |
|---|---|---|---|
| Cyclo (His-Pro) | [symbol] | [symbol] | [symbol] |
| Low Dose | | | |
| Cycle (His-Pro) | [symbol] | [symbol] | [symbol] |
| High Dose | | | |
| Cyclo (R-R*) | [symbol] | [symbol] | [symbol] |
| High Dose | | | |
| Inert agent | Control | X | X |

Certain experiments can use a wound-healing model. An excisional wound model can be generated by shaving and cleaning with 70% ethanol, the dorsal skin of mice (n=5 per group), which can be picked up at the midline and two layers of skin can be perforated with a sterile disposable biopsy punch (4 mm diameter; Miltex, York, Pa., USA), generating one wound on each side of the midline. After the excisional wound model is prepared, the process of wound healing will be observed for a predetermined number of days. In EPC transplantation experiments, after establishing the excisional wound model, EPCs (10⁵ cells) in 80 μl of PBS or 80 μl of PBS alone can be administered into the subcutaneous tissue around the wound defect. In certain asepcts the mice can be normal mice or mice with STZ-induced diabetes (e.g., n=5 per group). Each wound site will be documented at predetermined time points after injury, and wound areas determined by tracing the wound margins using the Image J software or similar method. The wound area at each time point can be calculated as a percent area of the original wound.

In certain aspects histological and immunohistological analysis can be performed. The wounds can be excised with the surrounding tissue. The tissue samples can be fixed with 4% paraformaldehyde in PBS at 4° C. for 24 h and embedded in paraffin to prepare histological or immunohistological slides. For histological analysis, tissue slices can be stained with hematoxylin and eosin (H&E) or Masson's trichrome dye. For immunohistological analysis, the slices can be incubated with anti-CD3 1, proliferating cell nuclear antigen (PCNA), cleaved caspase 3, alpha-smooth muscle actin, and vimentin antibodies (all from Santa Cruz Biotechnology, Dallas, Tex., USA) and followed by incubation with Alexa Fluor 488- or 594-conjugated secondary antibodies (Thermo Fisher Scientific, Waltham, Ma., USA). Nuclei can be stained with 4′,6-diamidino-2-phenylindole (Sigma-Aldrich). Immunostained slides were examined under confocal microscopy (Olympus, Tokyo, Japan). Each experiment was repeated at least three times.

Flow cytometric analysis can also be performed. To verify recruitment of the EPC population or leukocytes, wound tissues can be harvested and digested with 0.1% type II collagenase (Sigma-Aldrich) after postoperative day 3 or 7. EPCs or single cells derived from wound tissues can then be subjected to flow cytometric analysis using anti-Sca-1, anti-c-Kit, anti-Flk-1, anti-CD34, anti-CD3, anti-CD8, anti-CD11, and anti-CD45 antibodies (all from BD, San Jose, Calif., USA). Flow cytometry can be performed using a fluorescence-activated cell sorter (FACS; BD).

Cell proliferation can be assessed using the BrdU Cell Proliferation Assay Kit (Cell Signaling Technology, Beverly, Ma., USA) or Ez-CYTOX Kit (Daeil Biotech, Suwon, South Korea) according to the manufacturer's instructions.

In other aspects, tubule-like formation assays can be performed. To assess tubule-like formation capacity of EPCs, a Matrigel tube formation assay can be performed. Matrigel (BD) can be added to 96-well plates and incubated at 37° C. Cells (10⁴/well) can be seeded in Matrigel-coated plates and incubated for 6 h at 37° C. and 5% CO₂. The cells can be monitored by phase contrast microscopy (Olympus).

Cell migration assays can also be performed. For example cells can be plated in 6-well plates and grown until confluence and then the monolayer can be wounded with a cell scraper and the detached cells removed by gentle washing with the medium. Cells were incubated for 24 h at 37° C. and 5% CO₂ and examined under a microscope (Olympus) equipped with a×40 objective lens.

### REFERENCES

Hanselmann et al., “Haem oxygenase-1: a novel player in cutaneous wound repair and psoriasis?” Biochem. J., Vol. 353, 2001, pp. 459-466.
Hilton et al., “Cyclo(His-Pro) in nutritional supplements,” Lancet, Vol. 336, No. 8728, 1990, pg. 1455.
Hilton et al., Food contains the bioactive peptide, cyclo(His-Pro),” J. Clin. Endocrinol. Metab., Vol. 75, No. 2, 1992, pp. 375-378.
Minelli et al., “Cyclo(His-Pro) up-regulates heme oxygenase 1 via activation of Nrf2-ARE signaling,” J. Neurochem., Vol. 111, No. 4, 2009, pp. 956-966.
Uyemura et al., “Metabolism and Toxicity of High Doses of Cyclo (his-pro) Plus Zinc in Healthy Human Subjects,” J. Drug Metab. Toxicol. Vol. 1, No. 2, 2010, 1000105, 9 pages.

1. A wound care composition comprising an active ingredient comprising Cyclo-His-Pro.

2. The wound care composition of claim 1, wherein the composition further comprises a pharmaceutically acceptable carrier.

3. The would care composition of claim 1, wherein the composition is formulated for topical application onto a wound.

4. The wound care composition of claim 1, wherein the Cyclo-His-Pro is the only active ingredient.

5. The wound care composition of claim 1, wherein the composition further comprises an additional active ingredient selected from one or a combination of agents capable of moisturizing, increasing cellular uptake of compounds, increasing neovascularization, providing anti-microbial activity, elevating nitrogen monoxide levels, increasing Vascular Endothelial Growth Factor activity and/or concentration, enhancing re-epithelialization, and/or decreasing inflammation.

6. The wound care composition of claim 5, wherein the agent capable of increasing cellular uptake of compounds is a hydrogel, increasing neovascularization is a hydrogel, providing anti-microbial activity is Cyclo-Leu-Pro, Cyclo-Phe-Pro, chitosan, and/or zinc oxide, elevating nitrogen monoxide levels is arginine, increasing Vascular Endothelial Growth Factor activity and/or concentration is arginine, enhancing re-epithelialization is zinc oxide, and/or decreasing inflammation is zinc oxide.

7. The wound care composition of claim 1, wherein the composition further comprises one or more additional cyclic dipeptide(s).

8. The wound care composition of claim 7, wherein the one or more additional cyclic dipeptide(s) comprise proline as at least one of the two amino acids.

9. A method of treating a wound, the method comprising administering the composition of claim 1 to a subject having a wound.

10. The method of claim 9, wherein the composition of claim 1 is administered topically to the wound.

11. A method of reducing tissue damage in a wound, the method comprising administering the composition of claim 1 to a subject having a wound.

12. The method of claim 11, wherein the composition of claim 1 is administered topically to the wound.

13. A method of preventing and/or reducing oxidative damage in a wound, the method comprising administering the composition of claim 1 to a subject having a wound.

14. The method of claim 13, wherein the composition of claim 1 is administered topically to the wound.

15. A method of reducing the amount of heme in a wound, the method comprising administering the composition of claim 1 to a subject having a wound.

16. The method of claim 15, wherein the composition of claim 1 is administered topically to the wound.

17. A wound care composition comprising one or a combination of cyclic dipeptide(s) and a pharmaceutically acceptable carrier.

18. The wound care composition of claim 17, wherein the one or a combination of cyclic dipeptide(s) comprises proline as at least one of the two amino acids.

19. The wound care composition of claim 17, wherein the one or a combination of cyclic dipeptide(s) is one or a combination of: Cyclo-Ala-Pro; Cyclo-Arg-Pro; Cyclo-Asn-Pro; Cyclo-Asp-Pro; Cyclo-Cys-Pro; Cyclo-Glu-Pro; Cyclo-Gly-Pro; Cyclo-His-Pro; Cyclo-Ile-Pro; Cyclo-Leu-Pro; Cyclo-Lys-Pro; Cyclo-Met-Pro; Cyclo-Phe-Pro; Cyclo-Pro-Pro; Cyclo-Ser-Pro; Cyclo-Thr-Pro; Cyclo-Trp-Pro; Cyclo-Tyr-Pro; Cyclo-Val-Pro; and/or Cyclo-Gln-Pro.

20. The would care composition of claim 17, wherein the composition is formulated for topical application onto a wound.

21. The wound care composition of claim 17, wherein the composition further comprises an additional active ingredient selected from one or a combination of agents capable of moisturizing, increasing heme-scavenging, increasing cellular uptake of compounds, increasing neovascularization, providing anti-microbial activity, elevating nitrogen monoxide levels, increasing Vascular Endothelial Growth Factor activity and/or concentration, enhancing re-epithelialization, and/or decreasing inflammation.

22. The wound care composition of claim 21, wherein the agent capable of increasing heme-scavenging is Cyclo-His-Pro, increasing cellular uptake of compounds is a hydrogel, increasing neovascularization is a hydrogel, providing anti-microbial activity is Cyclo-Leu-Pro, Cyclo-Phe-Pro, chitosan, and/or zinc oxide, elevating nitrogen monoxide levels is arginine, increasing Vascular Endothelial Growth Factor activity and/or concentration is arginine, enhancing re-epithelialization is zinc oxide, and/or decreasing inflammation is zinc oxide.

23. A method of treating a wound, the method comprising administering the composition of claim 17 to a subject having a wound.

24. The method of claim 23, wherein the composition of claim 17 is administered topically to the wound.

25. A method of reducing tissue damage in a wound, the method comprising administering the composition of claim 17 to a subject having a wound.

26. The method of claim 25, wherein the composition of claim 17 is administered topically to the wound.

27. A method of preventing and/or reducing oxidative damage in a wound, the method comprising administering the composition of claim 17 to a subject having a wound.

28. The method of claim 27, wherein the composition of claim 17 is administered topically to the wound.

29. A method of reducing the amount of heme in a wound, the method comprising administering the composition of claim 17 to a subject having a wound.

30. The method of claim 29, wherein the composition of claim 1 is administered topically to the wound.

31. A method of reducing scarring of a wound, the method comprising administering the composition of claim 17 to a subject having a wound.
